(19) United States
(12) Patent Application Publication
(10) Pub. No.: US 2009/0263849 A1
(43) Pub. Date: Oct. 22, 2009

(54) Bioprinting Three-Dimensional Structure Onto Microscale Tissue Analog Devices for Pharmacokinetic Study and Other Uses

(75) Inventors: Wei Sun, Cherry Hill, NJ (US); Robert C. Chang, Cherry Hill, NJ (US); Binil Starly, Norman, OK (US); Jae Nam, Broomall, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: DREXEL UNIVERSITY, Philadelphia, PA (US)

(21) Appl. No.: 12/297,305

(22) PCT Filed: Apr. 23, 2007
(86) PCT No.: PCT/US2007/067210

§ 371 Date: Oct. 15, 2008

Related U.S. Application Data
(60) Provisional application No. 60/793,894, filed on Apr. 21, 2006.

(51) Int. Cl. C12Q 1/02; C12M 1/34

(52) U.S. Cl. 435/29; 435/287.3

(57) ABSTRACT

A microfluidic system for monitoring or detecting a change in a parameter of an input substance, which includes a microfluidic device having a tissue chamber and a tissue analog placed in the tissue chamber, wherein the tissue analog has a vessel structure mimicking naturally occurring vessel network incorporated in the tissue analog.

### BACKGROUND OF THE INVENTION

1. Field of Invention

This invention relates to a microfluidic system for monitoring or detecting a change in a parameter of an input substance. Specifically, the invention relates to a model for in vitro pharmacokinetic study and other pharmaceutical applications, as well as other uses such as computing, sensing, filtration, detoxification, production of chemicals and biomolecules, testing cell/tissue behavior, and implantation into a subject.

2. Description of Related Art

The existing technologies available and biomaterials employed are not currently amenable to creating 3-dimensional tissue analogs with high fidelity. Current practice also does not permit control of spatiotemporal placement of cells within a biomaterial matrix. Furthermore, current use of chemical coatings and modifications for cell/matrix attachment of microfluidic channels leads to residue formation and subsequent channel occlusions. Published biological data show that existing in vitro micro devices do not demonstrate good cell viability or preservation of normal in vivo cell-specific physiological function necessary to accurately perform pharmacokinetic studies on a long-term basis.

U.S. Pat. No. 6,916,640 Yu et al. describes culturing cells in a bioreactor using multi-layered microencapsulated cells.

U.S. Pat. No. 5,612,188 Shuler, et al. discloses a multi-chamber, in vitro system to simulate an interconnected organ system under a processor control. The system allows for gas exchange and fluid circulation. Within each chamber, cells of various types can be cultured which are representative of a desired organ. The multicompartmental cell culture system uses large components such as culture chambers, sensors, and pumps, which require the use of large quantities of culture media, cells and test compounds. This system is very expensive to operate and requires a large amount of space in which to operate. Because this system is on such a large scale, the physiological parameters vary considerably from those found in an in vivo situation. It is impossible to accurately generate physiologically realistic conditions at such a large scale.

U.S. Pat. No. 6,197,575 Griffith et al. describes a system for culturing cells using controlled channel structures to induce desired cell behavior and a sensing system to detect cellular or other environmental/material responses such as changes in metabolic products. A disadvantage of this system is that it still relies upon cell migration for cell seeding. There is no possibility for direct positional control of cell placement.

U.S. Pat. No. 6,133,030 Bhatia, et al. describes a method for positioning cells in patterns by surface modification of the substrate to promote cell-specific adhesion and then co-culturing a layer of cells on top of the cell-patterned layer. This could improve cell metabolic activity through more natural cell-cell interactions. However, this method is a 2-D cell patterning of the feeder layer. This method does not have the ability for 3-D positional control and patterning of cells.

U.S. Patent Application Publication 20070037275 to Shuler discloses a microscale cell culture device which comprises a fluidic network of channels segregated into discrete but interconnected chambers. The specific chamber geometry is designed to provide cellular interactions, liquid flow, and liquid residence parameters that correlate with those found for the corresponding cells, tissues, or organs in vivo. The fluidics are designed to accurately represent primary elements of the circulatory or lymphatic systems. In one embodiment, these components are integrated into a chip format. The design and validation of these geometries is based on a physiological-based pharmacokinetic model, a mathematical model that represents the body as interconnected compartments representing different tissues. The device can be seeded with the appropriate cells for each culture chamber. For example, a chamber designed to provide liver pharmacokinetic parameters is seeded with hepatocytes, and may be in fluid connection with adipocytes seeded in a chamber designed to provide fat tissue pharmacokinetics. The result is a pharmacokinetic-based cell culture system that represents the tissue size ratio, tissue to blood volume ratio, and drug residence time of the animal it is modeling.

This reference does not describe creating an artificial three dimensional tissue incorporated into a microfluidic device and therefore, it is limited to interactions of cells seeded on the surfaces of the chamber.

U.S. Patent Application Publication US20040259177A1 to Lowery described a high throughput screening system comprising a microfluidic device and a three-dimensional multicellular surrogate tissue assembly, wherein the cells are seeded within microbluiding channels which mimic laminar flow through naturally occurring tissue.

Despite the current developments, there is a need for a more efficient microfluidic system for monitoring or detecting a change in a parameter of an input substance in pharmacokinetic study and other applications.

All references cited herein are incorporated herein by reference in their entireties.

### BRIEF SUMMARY OF THE INVENTION

This invention is useful for pharmaceutical screening, bioassays, drug screening and discovery, predictive toxicology, drug metabolism and pharmacokinetics. It can also be used for a microscale in vitro static cell culture.

The invention includes a microfluidic system for testing drug metabolism in vitro, the microfluidic system comprises a microfluidic device having a bioprinted tissue made by a viable bioprinting solid freeform fabrication (SFF) process for a layer-by-layer deposition of a three-dimensional cell-encapsulated hydrogel-based tissue construct (see, for example PCT/US2004/015316 published as WO 2005/057436 for a description of the bioprinting SFF process), wherein the bioprinted tissue is integrated with a microfluidic device.

Further, the invention includes a method for testing drug metabolism in vitro, the method comprising providing the microfluidic system of the invention, providing a drug to be tested in a suitable medium, subjecting the microfluidic device to a flow mimicking conditions of a flow in a body of a mammal, collecting an output comprising a metabolite having a detectable parameter; and detecting the detectable parameter using techniques known in the art (e.g., fluorescence).

Accordingly, in one aspect, the invention is a microfluidic system for monitoring or detecting a change in a parameter of an input substance which includes (1) a microfluidic device, wherein the microfluidic device includes a microfluidic device, wherein the microfluidic device comprises (a) a cover platform having an inlet for delivery of an input substance and an outlet for removal of an output substance, (b) a substrate platform having (i) a tissue chamber in a shape of a depression in a substrate body of the substrate platform and (ii) a tissue analog having a vessel structure mimicking naturally occurring vessel network in a tissue analog three-dimensional construct comprising cells mixed with a tissue analog matrix, (c) a first microfluidic channel in fluid communication with the inlet for delivery of the input substance and the tissue chamber and (d) a second microfluidic channel in fluid communication with the outlet for removal of the output substance, provided that the substrate platform and the cover platform are superimposed to form a sealed assembly; and optionally (3) a pumping assembly and (4) a detecting unit.

In certain embodiments, the substrate platform comprises the first microfluidic channel and the second microfluidic channel in fluid communication with the tissue chamber.

In certain embodiments, the input substance is filled at least partially the vessel network of the tissue analog.

In certain embodiments, the cover platform comprises the first microfluidic channel and the second microfluidic channel in fluid communication with the tissue chamber.

In certain embodiments, at least one of the cover platform or the substrate platform comprises a surface with an improved hydrophilicity.

In certain embodiments, at least one of the cover platform or the substrate platform are made of a polymer, glass, a ceramic, a metal, an alloy, or a combination thereof.

In certain embodiments, the cover platform is made of a plasma treated glass and the substrate platform is made of a plasma treated biologically-compatible polymer composed of a plurality of siloxane units.

In certain embodiments, the tissue analog matrix comprises hydrogel.

In certain embodiments, the tissue analog is at least one of heart, stomach, kidney, intestine, lung, liver, fat, bone, cartilage, skeletal muscle, smooth muscle, cardiac muscle, bone marrow, muscle, brain, and pancreas.

In certain embodiments, the microfluidic system comprises a plurality of tissue chambers and microfluidic channels.

Another aspect of the invention is a method for monitoring or detecting a change in a parameter of an input substance, the method includes:

providing a microfluidic system of the invention as described above;

providing the input substance unit comprising the input substance;

directing the input substance into the microfluidic device, wherein the input substance flows through the inlet for delivery of the input substance and the first microfluidic channel into the vessel network in the tissue analog;

removing the output substance from the microfluidic device via the second microfluidic channel and the outlet for removal of the output substance;

obtaining at least a portion of the input substance prior to entry into the vessel network and at least a portion of the output substance after exiting the vessel network and thereby monitoring or detecting a change in the parameter of the input substance.

In certain embodiments of the method, the input comprises a drug and optionally a pharmaceutically acceptable carrier.

In certain embodiments of the method, said monitoring or detecting the change in the parameter of the input substance comprises collecting the output comprising a metabolite having a detectable parameter; detecting the detectable parameter; and correlating the detectable parameter to at least the extent and rate of metabolism.

Another aspect of the invention is a method of making the microfluidic system of the invention, the method comprising:

fabricating the cover platform comprising a cover body, an inlet port, an inlet opening, an outlet port, an outlet opening, and optionally microfluidic channels using microfabrication techniques;

fabricating the substrate platform comprising a substrate body, a tissue chamber, a first microfluidic channel and a second microfluidic channel wherein each microfluidic channel is in fluid communication with an input entry compartment and an output removal compartment, provided that each of the tissue chamber, the first microfluidic channel, the second microfluidic channel, the input entry compartment, and the output removal compartment represent indentations or depressions in the substrate body;

plasma treating the substrate platform and the cover platform;

making the tissue analog having the vessel structure mimicking naturally occurring vessel network in the tissue analog three-dimensional construct comprising cells mixed with the tissue analog matrix by using a bioprinting freeform fabrication process for a layer-by-layer deposition of the tissue analog matrix comprising cells;

forming the microfluidic device by superimposing the cover platform with the substrate platform such that the first microfluidic channel and the second microfluidic channel are in fluid communication with the tissue chamber, the an inlet port, the an outlet port, and the vessel structure; and

sealing the microfluidic device to provide the sealed assembly such that a flow of a substance can be conducted by engaging at least the inlet port, the first microfluidic channel, the second microfluidic channel, the vessel structure, and the outlet port and thereby making the microfluidic system.

Advantages of the invention over current methods include:
1. Computer-aided design (CAD) integration for structural reproducibility cell/biomaterial construct;
2. Reproducibility of 3-dimensional structures with low errors of margin between samples;
3. Biofriendly environment distinct from traditional harsh processing methods that require excessive pressure, heat, or toxic chemical agents;
4. Deposition capability of cell microencapsulating hydrogels provides biocompatible immunoisolation and accurately models in vivo physiology;
5. Printing capability for controlled spatiotemporal placement of cells within hydrogel;
6. Computer-aided design for high-fidelity reproducible structures, incorporation microencapsulating polymeric hydrogel biomaterial;
7. Three-dimensional structural formation capability; and
8. Biofriendly environment for sustained cell viability and cell-specific function.

### BRIEF DESCRIPTION OF SEVERAL VIEWS OF THE DRAWINGS

The invention will be described in conjunction with the following drawings in which like reference numerals designate like elements and wherein:

FIG. 1A is a scheme demonstrating a process of bioprinting a tissue-on-a-chip.

FIG. 1B is a scheme demonstrating a process of making the microfluidic device of the invention.

FIG. 2A is a top view of the microfluidic device of the invention without the tissue analog in the tissue chamber.

FIGS. 2B and 2C are side views demonstrating a step of making the tissue analog in a tissue chamber of the microfluidic device of the invention.

FIG. 3 is a top view of the microfluidic device of the invention with the tissue analog in the tissue chamber.

FIG. 4 is a scheme demonstrating a method for monitoring or detecting a change in a parameter of an input substance based on Fluorescent Microplate Reader analysis for determining a concentration of a drug and a metabolite.

FIG. 5A is a scheme demonstrating a design pattern for the tissue analog.

FIG. 5B is a scheme demonstrating a sandwich pattern for a tissue-on-a-chip application, a sample CAD model of a microfluidic chamber housing 3D microorgan.

FIG. 5C is a scheme demonstrating a sandwiched construct which simulates diffusion in all directions.

FIG. 6 is a micrograph demonstrating 3D structural feasibility and bioprinted tissue cell viability.

FIG. 7 is a bar graph demonstrating hepatocyte cell viability as a function of process parameters.

FIG. 8 is a bar graph demonstrating hepatocyte urea synthesis of 3D cell-encapsulated alginate versus 2D static cell culture.

### DETAILED DESCRIPTION OF THE INVENTION

The object of the invention is a new device and process for manufacturing such devices that reliably aids in the drug screening and drug discovery process. Additionally, the device will be able to perform metabolic and cytotoxicity studies on a microscale that is comparable to human physiologic scales. Faster drug screening methods with high-throughput capability and portability can lead to significant cost reductions attributed to reduced time and effort in the number of animal and human trial studies conducted. A suitable in vitro drug screening processes can aid in new drug discovery processes.

The invention further includes a method of making the microfluidic system. Furthermore, the fabrication process of bioprinting has been developed to build a 3-dimensional heterogeneous cell-encapsulated hydrogel-based construct within a microfluidic device which serves as a fluid circulator and as a platform for experimental drug/chemical analysis and toxicology.

The present invention represents an in vitro model that can realistically predict human response to various drug administrations and toxic chemical exposure. By fabricating a three-dimensional in vitro tissue analog comprising an incorporated array of microfluidic channels and tissue-embedded chambers, one can selectively biomimic different mammalian tissues for a multitude of applications, e.g., a liver tissue for experimental pharmaceutical screening of drug efficacy and toxicity. A rational approach for the reconstruction of such an in vitro model is 1) the development of a viable bioprinting freeform fabrication process for making a bioprinted tissue by, for example, a layer-by-layer deposition of a three-dimensional cell-encapsulated hydrogel-based tissue construct and 2) the direct printing of the tissue construct onto a plasma surface-treated microfluidic device.

Accordingly, in one aspect, the invention is a microfluidic system for monitoring or detecting a change in a parameter of an input substance which includes (1) a microfluidic device, wherein the microfluidic device includes a microfluidic device, wherein the microfluidic device comprises (a) a cover platform having an inlet for delivery of an input substance and an outlet for removal of an output substance, (b) a substrate platform having (i) a tissue chamber in a shape of a depression in a substrate body of the substrate platform and (ii) a tissue analog having a vessel structure mimicking naturally occurring vessel network in a tissue analog three-dimensional construct comprising cells mixed with a tissue analog matrix, (c) a first microfluidic channel in fluid communication with the inlet for delivery of the input substance and the tissue chamber and (d) a second microfluidic channel in fluid communication with the outlet for removal of the output substance, provided that the substrate platform and the cover platform are superimposed to form a sealed assembly; and optionally (3) a pumping assembly and (4) a detecting unit.

Inventors have discovered that a tissue analog having a desired micro vessel structure can be directly printed into a tissue chamber's indentation (a depression) created using soft lithographic techniques (e.g., nanotransfer printing, microtransfer molding, replica molding, micromolding in capillaries, near field phase shift lithography, and solvent assisted micromolding; see, for example U.S. Pat. No. 7,195,733 to Rogers et al.) and used as a flow mimicking reservoir thus replacing the previously described microchannels seeded with cells.

MEMS microfabrication is a useful process for biochip fabrication and simulating microflow conditions, however is not yet accepted for integrating cells into the process directly. Cells are generally seeded after fabrication of the microfluidic system to grow within the microchannels. SFF can create complex 3-D shapes, and deposit biomaterials and cells for tissue engineering, but it is not as useful as MEMS microfabrication in incorporating complex electromechanical elements, actuators, and valves to create microflow systems.

Advantageously, the inventor have combined the two processes to provide much greater benefit than either process by itself and overcomes the limitations of either method. SFF can be used to deposit/seed cells directly into channels or other positional locations within the microfluidic device and build tissue constructs within chambers that exhibit spatial patterning.

Bioprinting System (SFF techniques used include, but not limited to, 3DP, syringe dispensing, piezoelectric glass capillary jetting, thermal and ink-jetting, solenoid valve-based jetting, polymer-based UV curing, deposition, and sprays).

Biofriendly Environment—No use of excessive pressure, heat or toxic chemicals.

Cell Encapsulation and Printing Capability.

CAD Integration to produce complex 3D patterns.

Multi-Nozzle capability in producing heterogeneous tissue constructs.

Reproducibility of 3D structures with low errors of margin among samples.

Several different layered manufacturing capabilities have been developed to produce an artificial tissue. Among them is the multinozzle bioprinting system capable of depositing different biomaterials and cells reproducibly in precise locations (see PCT/US2004/015316 published as WO 2005/057436).

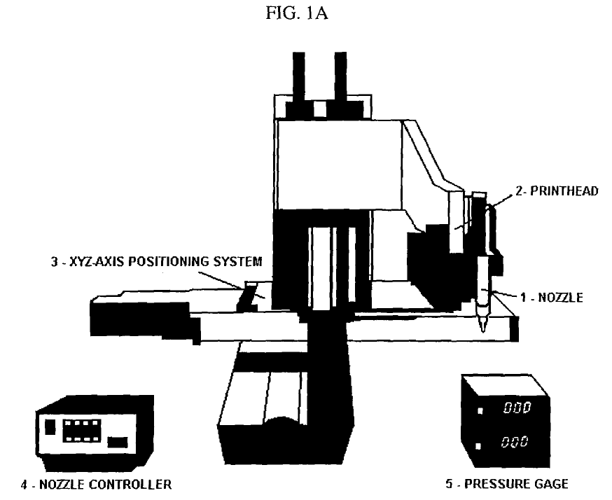

A single nozzle bioprinting system can also be used. An exemplary embodiment of a bioprinting system is illustrated in FIG. 1A (front view), it consists of one or more nozzles 1 mounted on a printhead 2. The printhead 2 is attached to a computer-controlled xyz-positioning system 3. Dispensing of material is handled by the nozzle controller 4. Gages 5 are used to monitor process parameters such as pressure.

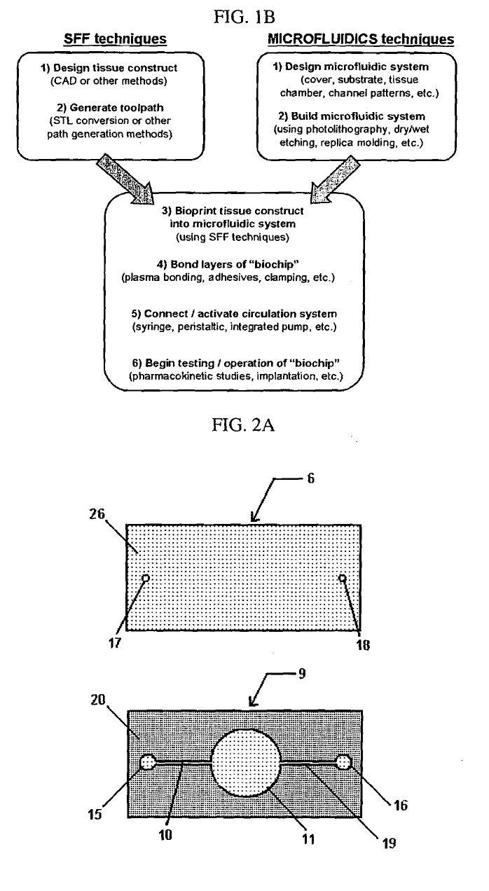

The bioprinting system is used to build 3-D tissue constructs within a microfluidic system (see FIG. 1B) as shown in FIGS. 2A-2C. FIG. 2A shows a basic, 2-platform embodiment of a microfluidic device of the microfluidic system of the invention.

The term “tissue-on-a chip” as used herein means the microfluidic device of the invention wherein the tissue analog is bioprinted into a chamber located in the substrate platform, which is joined with a cover platform to form the microfluidic device in a shape of a chip.

The term “bioprinting” as used herein means a process of making a tissue analog by depositing scaffolding (matrix) material mixed with cells based on computer driven mimicking of a texture and a structure of a naturally occurring tissue.

FIG. 2A is a top view of the microfluidic device of the invention. It comprises two major units: a cover platform 6 and a substrate platform 9 which are superimposed and are held together by various ways, such as, for example an assembly of a screw and a nut. In a preferred embodiment, no additional means for holding the platforms are required; having been plasma treated, the two platforms can form a strong irreversible bond to prevent leaking.

The cover platform 6 comprises a cover body 26, an inlet port 7 and an outlet port 8 located on opposite sides of the cover platform 6, an inlet opening 17 (FIG. 2C) and an outlet opening 18 (FIG. 2C) attached to or integrated with corresponding inlet port 7 and an outlet port 8 positioned on opposite sides of the cover body 6 such that the inlet opening 17 and the outlet opening 18 are positioned on the top portion of the cover body 6 and superimposed with the inlet port 7 and the outlet port 8; tubing 14 connected to the inlet opening 17 and the outlet opening 18 for delivery of an input medium and removal of an output medium. It should be understood that the inlet port and the outlet port can have different shapes which are not limited to a cylinder shape; the ports can also be integrated as a single unit with the corresponding opening as well as with corresponding tubing.

The cover body can be manufactured from glass or other suitable materials, a polymer, ceramic, metal, alloy, or any combination thereof. In a preferred embodiment, the cover body is made of glass. It is preferred that the glass or other suitable materials are plasma treated to provide improved hydrophilicity. Methods of plasma treatment are known in the art, see, for example U.S. Pat. No. 6,967,101 to Larsson et al and U.S. Pat. No. 5,028,453 to Jeffrey et al.

The substrate platform 9 comprises a substrate body 20, a tissue chamber 11, a microfluidic channel 10 for an input media (a first microfluidic channel) and a microfluidic channel 19 for an output media (a second microfluidic channel) wherein each microfluidic channel is connected with an input entry compartment 15 and an output removal compartment 16. The input entry compartment 15 and the output removal compartment 16 are indentations or depressions in the substrate body 20 which are designed to assure smooth flowing of both input and output substance delivered from the inlet port 7 and removed from the outlet port 8. The input entry compartment 15 and the output removal compartment 16 can be deeper and/or wider than the microfluidic channels they are connected to. The microfluidic channels are etched or otherwise indented conduits which provide a delivery route for an input medium to the tissue analog located in the tissue chamber 11 and a removal route for the output medium from the tissue analog.

In certain embodiments, the delivery route for an input medium and the removal route for the output medium can be modified such that the microfluidic channels are etched in the cover body or partially etched in the cover body and partially etched in the substrate body. It should be understood that the purpose of the microfluidic channels is to deliver and remove the medium to and from the tissue analog in a closed assembly of the cover platform and the substrate platform.

In certain embodiments, the microfluidic systems have various shapes of the tissue chamber, e.g., square, oval, irregular, etc. In certain embodiments, a square tissue chamber is etched in the substrate platform; microchannels are etched in the glass platform and direct flow into the tissue chamber on the bottom layer.

The tissue chamber 11 is located approximately in the middle of the substrate body 20. More than one tissue chamber can be utilized in the same substrate body. In certain embodiments, multiple tissue chambers would have an independent set of input/output routes; in other embodiments, several tissue chambers can be placed consecutively one after another and utilize various input/output routes or a single input/output route.

The substrate can be manufactured from the following exemplary materials: a polymer, ceramic, glass, metal, alloy, or any combination thereof. In preferred embodiments, the polymer comprises a biologically-compatible polymer. Suitable biologically-compatible polymers include a plurality of units derived from a siloxane, an alkyl oxide such as ethylene oxide, an acrylic, an amide, a polymerizable carboxylic acid group, or any combination thereof. When the biologically-compatible polymers include a plurality of units derived from a siloxane, the siloxane units typically include a plurality of monomers that include dimethyl siloxane, or any combination thereof. A preferred biologically-compatible polymer composed of a plurality of siloxane units is polydimethyl siloxane (“PDMS”). Any other type of polymeric material that can be fabricated into optically transparent microfluidic devices, for example polymethylmethacrylate (“PMMA”), can also be used. The substrate material has to meet the primary requirement of biocompatibility and hydrophilicity. It is preferred that the substrate materials are plasma treated to provide permanent bonding as well as improved hydrophilicity for the PDMS substrate.

The cover body and substrate body materials that are not necessarily biologically-compatible can also be used in some embodiments of the present invention. In these embodiments, substrate materials that are not alone biologically-compatible can be made compatible using a suitable surface treatment or coating to make them biologically-compatible. Suitable surface treatments or coatings can include a thin film of a biologically-compatible material applied to the surface of a typically biologically-incompatible substrate. For example, the microfluidic structures patterned in a biologically-incompatible substrate can be surface treated with an optional adhesion modifying agent and then coated with a thin film of a biologically-compatible material, such as PDMS.

Making indentation or etchings in the substrate can be done by methods known in the art, for example dry etching techniques such as deep-reactive ion etching, wet etching techniques using acids, and replica molding techniques. PDMS base and curing agent can be poured into a mold, degassed under vacuum, and then heated to create the PDMS platform.

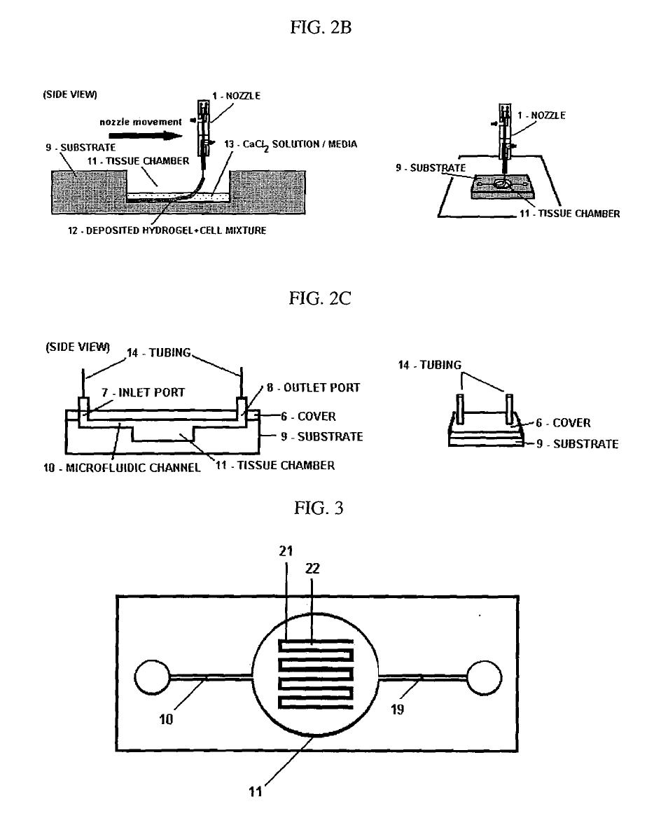

Tissue chamber 11 is designed to serve as a compartment or a “mold” for a tissue analog 21 with a pattern of inner vessels 22 which mimics a pattern of naturally occurring vessels as shown in FIG. 3. FIG. 3 is a top view of the microfluidic device of the invention with the tissue analog in the tissue chamber. The tissue analog is deposited from a nozzle of a bioprinting device which is operated based on computerized calculations and allows mimicking a desired tissue as a three dimensional construct. An exemplary bioprinting device is described in PCT/US20041015316 published as WO 2005/057436 and in US Patent Application Publication US 2006/0105011), incorporated herein in its entirety. The bioprinting material can be a biopolymer, preferably hydrogel, such as, for example, alginate, which is mixed with cells known to be present in a particular tissue or other cells depending on a desired application. Thus, a three dimensional tissue analog is bioprinted directly in the tissue chamber 11. Depending on the design of the experiment for measuring and analyzing output, there may be an empty space left in the tissue chamber; preferably, the tissue chamber is filled entirely.

Upon completion of printing, the top and bottom layers are bonded together as shown in FIG. 2C. For this embodiment, cleaning of the two surfaces to be joined is done with 70% ethanol, acetone, and deionized water, then plasma treatment is used to bond the cover 6 to the substrate 9. For hydrophobic materials such as PDMS, plasma treatment can be done prior to bioprinting to improve surface hydrophilicity, wettability, and cell adhesion within the tissue chamber and microchannels.

The input substance is administered to the tissue analog 21 through the tubing 14, the inlet port 7, the inlet opening 17, the input entry compartment 15, and the microfluidic channel 10. The input substance can be administered with the help of a pump (not shown) or gravity forces. A pump (e.g., syringe pump, peristaltic pump, microfluidic pumps, etc.) can be used at a calculated flow rate for desired residence time or shear flow. The pressure created in the device of the invention should be monitored to ensure that the flow is achieved and the seal is not compromised or the tissue adversely affected.

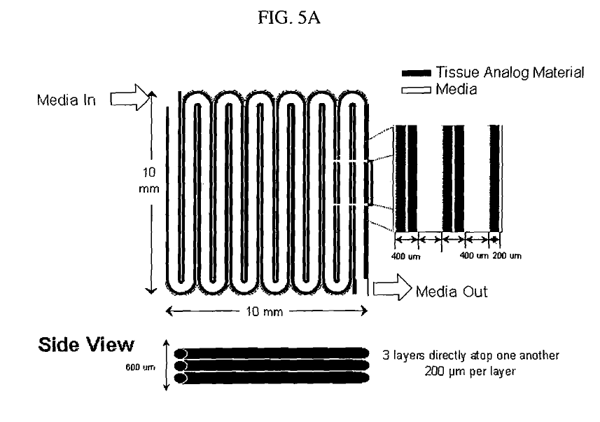

Once the input medium reached the tissue chamber 11 and the tissue analog 21, the input medium finds its way through the inner vessels 22 (see FIG. 5A) and exits as an output into the microfluidic channel 19, the output removal compartment 16, the outlet opening 18, the outlet port 8, and the tubing 14. The output is then collected and analyzed for a change in a selected parameter of the tested material such as, for example, for metabolic activity or for reaction end products. Such analysis is conducted using methods well known in the art. Suitable assays involve measuring a change in a selected parameter such as, for example, absorbance, fluorescence or nuclear magnetic resonance (NMR) properties of reporter molecules in a high throughput screening mode in 24, 48, or 96 well format currently used for drug candidate screening. It is envisioned that biochemical assay reporter molecules can be introduced into the microfluidic culture channels or produced by cells in the bioprinted tissue analog and direct measurements of change in the reporter molecule could be taken directly from the microfluidic device. This may provide a rapid method for verifying that compounds showing desired biochemical properties during initial screening and a corresponding inhibition or promotion of cell development are actually functioning as predicted.

Further, a morphological analysis may be carried out using an inverted microscope; fluorescence labeling of cells, organelles, or macromolecules using exogenous fluors or expressed fluorescent proteins, such as green fluorescent protein, may be useful for detecting changes in cell properties. Enzyme linked immunosorbent assays (ELISA) may be used to determine the presence or quantity of, for example, growth factors. Metallo-proteases are often an indicator of tissue differentiation or tissue invasion and Zymogram gels (Invitrogen, Carlsbad, Calif.) are useful in measuring this activity.

This embodiment in FIG. 2B shows two different side views of the nozzle 1 depositing a hydrogel mixed with a cell mixture 12 into a CaCl₂ crosslinking solution plus cell media 13 onto the substrate layer 9 within the tissue chamber 11. Complex patterns and structures can be created in this way through a layer-by-layer fashion.

Finally, tubing 14 is connected to the inlet 7 and outlet 8 ports.

Method of Making Microfluidic Systems

Another aspect of the invention is a method of making the microfluidic system of the invention, which includes

(a) fabricating the cover platform comprising a cover body, an inlet port, an inlet opening, an outlet port, an outlet opening, and optionally microfluidic channels using microfabrication techniques;

(b) fabricating the substrate platform comprising a substrate body, a tissue chamber, a first microfluidic channel and a second microfluidic channel wherein each microfluidic channel is in fluid communication with an input entry compartment and an output removal compartment, provided that each of the tissue chamber, the first microfluidic channel, the second microfluidic channel, the input entry compartment, and the output removal compartment represent indentations or depressions in the substrate body;

(c) plasma treating the substrate platform and the cover platform;

(d) making the tissue analog having the vessel structure mimicking naturally occurring vessel network in the tissue analog three-dimensional construct comprising cells mixed with the tissue analog matrix by using a bioprinting freeform fabrication process for a layer-by-layer deposition of the tissue analog matrix comprising cells;

(e) forming the microfluidic device by superimposing the cover platform with the substrate platform such that the first microfluidic channel and the second microfluidic channel are in fluid communication with the tissue chamber, the an inlet port, the an outlet port, and the vessel structure; and

(f) sealing the microfluidic device to provide the sealed assembly such that a flow of a substance can be conducted by engaging at least the inlet port, the first microfluidic channel, the second microfluidic channel, the vessel structure, and the outlet port and thereby making the microfluidic system.

Microfabrication techniques such as photolithography, etching of silicon and glass, or replica molding and soft lithography techniques are well established in the literature, and can be used to create a wide variety of microfluidic systems.

As part of the process development phase, experiments were done testing multinozzle heterogeneous printing using a complex, multi-material part in CAD. For example, simultaneously deposited were materials containing different alginate solutions admixed with cells and biological factors ionically crosslinked for structural optimization and integrity. Three-dimensional hydrogel scaffolds have also been extruded as an alginate filament with the nozzle tip submerged within a crosslinking solution. The power of computer-aided design techniques is recruited to create hydrogel tissue constructs with various patterns. In order to ensure compatibility with a microscale cell culture analog system, boundary studies have been carried out with alginate testing the potential limits and capabilities of the bioprinting system, resulting in the creation of filaments within the 30-40 micron diameter range.

The candidate materials selected for the bioprinting of tissue constructs must meet the criteria of a polymeric hydrogel, biocompatible, and biodegradable. Although other polymeric materials may be extruded using our bioprinting process, the overwhelming experience and work with similar pneumatic-driven, syringe-based systems hitherto have employed the use of hydrogels. Hydrogels are useful biomaterials for 3D cell culture because of their high water content and mechanical properties resemble those of tissues in the body. While the dual criteria of biocompatibility and biodegradability of tissue construct materials are obligatory, it does not naturally translate into good cell viability (i.e. comparable to static two-dimensional cell culture) and physiological tissue function. One candidate hydrogel polymer that has demonstrated good cell viability and cell-specific function with the bioprinting process is sodium alginate, a co-block polysaccharide natural biopolymer.

Micro-scale tissue analog (e.g., a liver or other desired tissue) are designed and fabricated via direct deposition of a three dimensional heterogeneous cell-seeded hydrogel-based matrix. By integrating the bioprinting system with a CAD environment, notable feasibility and reproducibility of 3D structures within micron-order dimensional specifications have been realized. Repeated testing has also demonstrated good cell viability and maintenance of liver cell-specific function for post-assembly bioprinted encapsulated hepatocytes (liver cells) under biofriendly conditions using Live/Dead cell assays, Alamar Blue staining with cytofluorimetry, and functional bioassays.

A Method for Monitoring or Detecting a Change in a Parameter of an Input Substance

Another aspect of the invention is a method for monitoring or detecting a change in a parameter of an input substance which includes:

(a) providing a microfluidic system of the invention as described above;

(b) providing the input substance unit comprising the input substance;

(c) directing the input substance into the microfluidic device, wherein the input substance flows through the inlet for delivery of the input substance and the first microfluidic channel into the vessel network in the tissue analog;

(d) removing the input substance from the microfluidic device via the second microfluidic channel and the outlet for removal of the output substance; and

(e) obtaining at least a portion of the input substance prior to entry into the vessel network and at least a portion of the output substance after exiting the vessel network and thereby monitoring or detecting a change in the parameter of the input substance.

A Model for Pharmacokinetic Study of the Invention

Since hepatocytes (liver cells) are the cells that steward the metabolic and biosynthetic processes in the body, bioprinted liver tissue constructs will be an exemplary chamber/compartment in microfluidic circuits.

By combining SFF with microfluidics, an in vitro circulating system of drug perfusate is constructed for liver tissue construct functional analysis. Liver tissue is used herein as an example and should not be interpreted as a limitation to the invention as any tissue analog can be used in this invention.

Existing kinetic and thermodynamic equations may be written for each tissue construct/organ analog that describe the behavior of a drug or chemical in that organ. For example, in the liver compartment of a tissue-on-a-chip microdevice, a model drug compound is in large part metabolized by the cytochrome P450 monooxygenase system (CYP450) into reactive metabolites. Notably, clearance is the most important parameter in pharmacokinetics and provides a suitable basis for quantitative evaluation and comparison of fabricated liver tissue constructs with that of a normal human liver. The clearance of a drug is the volume of body fluid inflow from which the drug is completely removed by biotransformation and/or excretion, per unit time. Clearance is a pharmacokinetic parameter which is experimentally evaluated as a function of varying design parameters and biomaterial properties and subsequently optimized.

Rₘ=CLC₁

Mass Conservation Law:

$${V}_{1} \frac{d {C}_{1}}{d t} = - {QC}_{1} + {QC}_{2} + R$$

$${V}_{2} \frac{d {C}_{2}}{d t} = - {QC}_{2} + {QC}_{1} - {CLC}_{1}$$

CL: volume of the inflow to the tissue analog from which the drug would be entirely removed in unit time
Rm: rate of metabolism in tissue analog
Q: circulating rate of perfusate
C₁: drug concentration entering tissue analog
C₂: drug concentration exiting tissue analog
R: constant rate of continuous infusion
D: Total amount of Drug in the medium

Initial Conditions:

$${C}_{1} {\vert}_{t = 0} = \frac{D}{{V}_{1}} , \\ {C}_{2} {\vert}_{t = 0} = 0 , \\ R = 0$$

CL is then obtained from the following relation:

$$\frac{A}{α} + \frac{B}{β} = \frac{D}{CL}$$

CL is dependent on

$$CL = \frac{D α β}{β A + α B}$$

D—amount of drug which in turn relates to Cl
α, β—slope of graph which is dependent on cell density and cell type, biomaterial properties
A,B—intercept values of graph which is dependent on
   Q=Flow rate of perfusate (medium+drug)
   V2=Flow Volume of Construct Channel
      (Length×Cross Sectional Area)

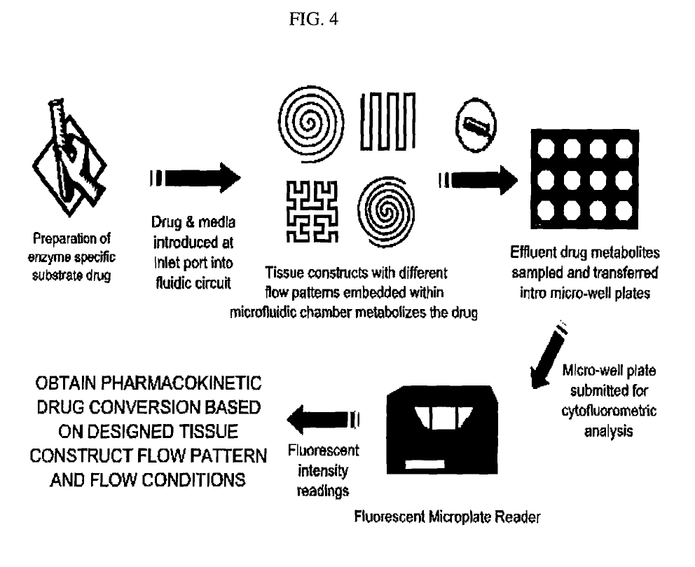

To demonstrate an effective drug metabolism in the model system, a non-fluorescent prodrug is fed into system, metabolized by the liver chamber, and a fluorescent metabolite produced therein is analyzed for relative fluorescent intensity, which is directly proportional to the relative drug metabolite concentration (FIGS. 3 and 4). Such an analysis will prove information regarding the relative pharmacokinetic efficiency and relevancy of the microfabricated tissue-on a-chip of the invention for human application.

FIG. 4 shows a scheme demonstrating a process of Fluorescent Microplate Reader analysis for determining a concentration of a drug and a metabolite, wherein a mixture of a drug and a media is introduced at an inlet port into a fluidic circuit of a tissue construct of the invention with has a flow pattern of channels embedded within a microfluidic chamber. It should be understood that a flow pattern of channels can vary and is not limited to the patterns depicted in FIG. 4.

Effluent drug metabolites are collected on micro-well plates to be tested in a Fluorescent Microplate Reader in accordance with known techniques.

Design of a Three-dimensional Tissue Analog within a Tissue Chamber of a Microfluidic Device.

In certain embodiments, the microfluidic device of the invention is created using microfabrication techniques. For example, to create a polydimethosiloxane (PDMS) microfluidic device, a mold with microfluidic channels and a tissue chamber can be fabricated using photolithography with a negative photoresist such as SU-8. PDMS base and curing agent can be poured into the mold, degassed under vacuum, and then heated to create the PDMS layers or a platform.

The PDMS substrate is surface modified using, for example, air plasma treatment, to facilitate direct bioprinting. The substrate is placed within an RF plasma cleaner with vacuum applied for a minute to evacuate the chamber. The PDMS substrate is then exposed to the RF plasma for 30 seconds to improve surface hydrophilicity and adhesion properties to glass and surface treated PDMS.

Hydrogel (e.g., alginate)-encapsulated cells are then printed into the tissue chamber of the plasma-treated substrate using solid freeform fabrication (SFF) techniques in accordance with computer driven structure of a desired tissue analog. The model for the desired tissue structure is created on a computer and converted into a readable format for the xyz-axis motion control system. Process parameters such as printhead speed, pressure, nozzle inner diameter, temperature, and solution viscosity can be set depending upon the desired properties of the tissue construct.

Upon completion of the printing process, the two platforms are joined (e.g., adhered, bonded, or otherwise connected) together. Having been plasma treated, the two layers can form a strong irreversible bond to prevent leaking.

The sealed microfluidic device containing the 3-D tissue analog is then connected to a syringe pump for controlled simultaneous infusion of a testing substance (e.g., a drug in an appropriate medium) at the inlet port and withdrawal at the outlet port.

In certain embodiment, the pattern of the tissue analog can vary. As shown in FIGS. 5A-C, the alginate construct pattern is sandwiched in between at least 2 alginate construct beds. The construct pattern can be created in a CAD environment (in silico), converted to an STL file and then converted into a toolpath. This toolpath can be used by the motion control software to direct the printhead and create the desired part. Alternatively, for a simple design, the toolpath can be created directly by using the motion control programming software. The ability to vary the geometry within the tissue chamber is one of the main advantages to combining SFF with microfluidics. The pattern can be as simple or as complex as desired. A standard biochip could be mass produced but could be tailored to many different functions by simply printing different constructs/patterns/cell types within the tissue chamber.

In FIG. 5B, the 3 alginate layers represent the layered bioprinting fabrication approach to produce 3D tissue constructs within the chamber. Depending on the flow pattern specifications, the process toolpath leads to different patterns for each layer as well as orientation of each subsequent layer with respect to the preceding layer.

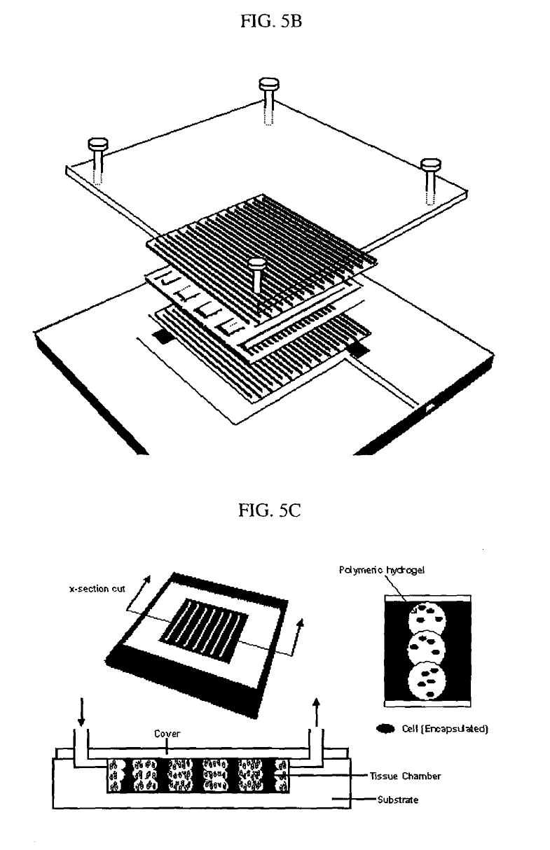

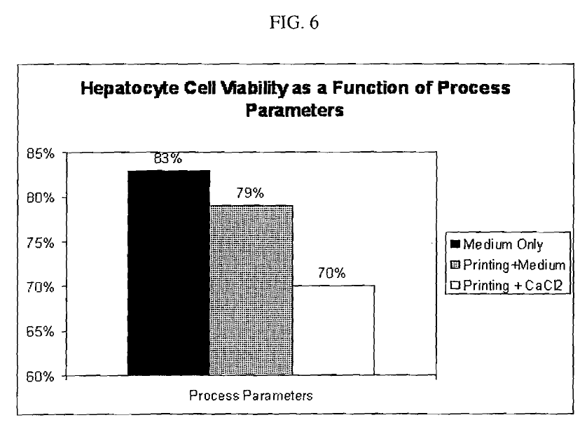

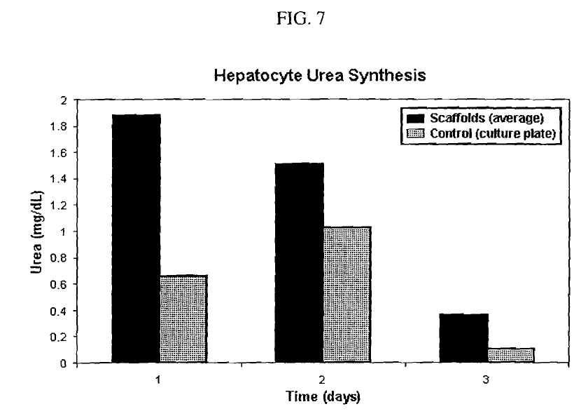

As shown in FIGS. 6-8, preliminary cell viability tests of the pneumatic printing process demonstrate that hepatocytes were able to survive with a 79% cell viability ratio. Hepatocytes encapsulated in alginate synthesized a higher amount of urea than the same number of hepatocytes cultured on tissue culture plastic (TCP).

Other embodiments of the device/process could substitute different materials for the substrate such polymers, rubbers, plastics, metals, etc. depending upon the desired mechanical, electrical, biological, or other properties such as material strength, conductivity, cell adhesion, biocompatibility, or optical transparency/opacity.

In certain embodiments, glass layers can be used instead of or in addition to alginate layers. Also chrome layers can be deposited as a mask with photolithography used to expose the desired channel pattern to be used as masks, or metal layers to be used as electrodes. Wet etching with fluoridic acid (HF) would create channel structures in the glass. Such techniques could be combined with abrasive operations using diamond-coated bits. For silicon layers, MEMS techniques such as deep RIE, wet etching, and other standard industry techniques can be used to create the desired geometry.

In other embodiments, the substrate platform can be modified in different ways such as plasma treatment, alterations in surface charge, covalent bonding of proteins and other moieties, surface roughening, oxidation, etching, and other surface modification procedures to create the desired properties, e.g., better cell adhesion, wetting properties, etc.

Alternative embodiments could vary the bonding method of the layers such as chemical modification or the use of adhesives. Additionally, many multiple layers could be bonded together to form stacks of biochips.

Alternative embodiments may deliver the cells using non-gels such as liquid media, solids, or gases, and does not necessarily require cell encapsulation.

Additional embodiments may not even use cells but deposit acellular material. For example, micelles, plasma membrane analogues, or other non-living components could be deposited for pharmacokinetic or other studies.

Other embodiments of the microfluidic system further include incorporating electrodes for directed electroosmotic and electrokinetic flow, or for heating, temperature regulation, and sensor functions, and also the incorporation of microvalves and micropumps known in the literature [4, 5].

The device of the invention may include a mechanism for obtaining signals from the cells of the tissue analog and/or the medium. The signals from different chambers and channels can be monitored in real time. For example, biosensors can be integrated or external to the device, which permit real-time readout of the physiological status of the cells in the system.

Any cell type is suitable for use with this invention, such as for example, primary cells, stem cells, progenitor cells, normal, genetically-modified, genetically altered, immortalized, and transformed cell lines, single cell types or cell lines, or with combinations of different cell types. Preferably, the cultured cells maintain the ability to respond to stimuli that elicit a response in their naturally occurring counterparts. These may be derived from all sources such as eukaryotic or prokaryotic cells. The eukaryotic cells can be plant, or animal in nature, such as human, simian, or rodent. They may be of any tissue type (e.g., heart, stomach, kidney, intestine, lung, liver, fat, bone, cartilage, skeletal muscle, smooth muscle, cardiac muscle, bone marrow, muscle, brain, pancreas), and cell type (e.g., epithelial, endothelial, mesenchymal, adipocyte, and hematopoietic).

In addition, cells that have been genetically altered or modified so as to contain a non-native “recombinant” (also called “exogenous”) nucleic acid sequence, or modified by antisense technology to provide a gain or loss of genetic function may be utilized with the invention. Methods for generating genetically modified cells are known in the art, see for example “Current Protocols in Molecular Biology,” Ausubel et al., eds, John Wiley & Sons, New York, N.Y., 2000. The cells could be terminally differentiated or undifferentiated, such as a stem cell. The cells of the present invention could be cultured cells from a variety of genetically diverse individuals who may respond differently to biologic and pharmacologic agents. Genetic diversity can have indirect and direct effects on disease susceptibility. In a direct case, even a single nucleotide change, resulting in a single nucleotide polymorphism (SNP), can alter the amino acid sequence of a protein and directly contribute to disease or disease susceptibility. For example, certain APO-lipoprotein E genotypes have been associated with onset and progression of Alzheimer's disease in some individuals.

Drugs, toxins, cells, pathogens, samples, etc., herein referred to generically as “input variables” are screened for biological activity by adding to the pharmacokinetic-based culture system, and then assessing the cultured cells for changes in output variables of interest, e.g., consumption of O₂, production of CO₂, cell viability, or expression of proteins of interest. The input variables are typically added in solution, or readily soluble form, to the medium of cells in culture. The input variables may be added using a flow through system, or alternatively, adding a bolus to an otherwise static solution. In a flow-through system, two fluids are used, where one is a physiologically neutral solution, and the other is the same solution with the test compound added. The first fluid is passed over the cells, followed by the second. In a single solution method, a bolus of the test input variables is added to the volume of medium surrounding the cells. The overall composition of the culture medium should not change significantly with the addition of the bolus, or between the two solutions in a flow through method.

Preferred input variables formulations do not include additional components, such as preservatives, that have a significant effect on the overall formulation. Thus, preferred formulations include a biologically active agent and a physiologically acceptable carrier, e.g., water, ethanol, or DMSO. However, if an agent is liquid without an excipient, the formulation may be only the compound itself.

Preferred input variables include, but are not limited to, viruses, viral particles, liposomes, nanoparticles, biodegradable polymers, radiolabeled particles, radiolabeled biomolecules, toxin-conjugated particles, toxin-conjugated biomolecules, and particles or biomolecules conjugated with stabilizing agents. A “stabilizing agent” is an agent used to stabilize drugs and provide a controlled release. Such agents include albumin, polyethyleneglycol, poly(ethylene-co-vinyl acetate), and poly(lactide-co-glycolide).

A plurality of assays may be run in parallel with different input variable concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection.

Input variables of interest encompass numerous chemical classes, though frequently they are organic molecules. A preferred embodiment is the use of the methods of the invention to screen samples for toxicity, e.g., environmental samples or drug. Candidate agents may comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Included are pharmacologically active drugs and genetically active molecules. Compounds of interest include chemotherapeutic agents, anti-inflammatory agents, hormones or hormone antagonists, ion channel modifiers, and neuroactive agents. Exemplary of pharmaceutical agents suitable for this invention are those described in “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming Organs; Hormones and Hormone Antagonists; Vitamins, Dermatology; and Toxicology, all incorporated-herein by reference. Also included are toxins, and biological and chemical warfare agents, for example see Somani, S. M. (Ed.), “Chemical Warfare Agents,” Academic Press, New York, 1992).

Test compounds include all of the classes of molecules described above, and may further comprise samples of unknown content. While many samples will comprise compounds in solution, solid samples that can be dissolved in a suitable solvent may also be assayed. Samples of interest include environmental samples, e.g., ground water, sea water, or mining waste; biological samples, e.g., lysates prepared from crops or tissue samples; manufacturing samples, e.g., time course during preparation of pharmaceuticals; as well as libraries of compounds prepared for analysis; and the like. Samples of interest include compounds being assessed for potential therapeutic value, e.g., drug candidates from plant or fungal cells.

The term “samples” also includes the fluids described above to which additional components have been added, for example, components that affect the ionic strength, pH, or total protein concentration. In addition, the samples may be treated to achieve at least partial fractionation or concentration. Biological samples may be stored if care is taken to reduce degradation of the compound, e.g., under nitrogen, frozen, or a combination thereof. The volume of sample used is sufficient to allow for measurable detection, usually from about 0.1 micron to 1 ml of a biological sample is sufficient.

Compounds and candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, naturally or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification to produce structural analogs.

Output variables: Output variables are quantifiable elements of cells, particularly elements that can be accurately measured in a high throughput system. An output can be any cell component or cell product including, e.g., viability, respiration, metabolism, cell surface determinant, receptor, protein or conformational or posttranslational modification thereof, lipid, carbohydrate, organic or inorganic molecule, mRNA, DNA, or a portion derived from such a cell component. While most outputs will provide a quantitative readout, in some instances a semi-quantitative or qualitative result will be obtained. Readouts may include a single determined value, or may include mean, median value or the variance. Characteristically a range of readout values will be obtained for each output. Variability is expected and a range of values for a set of test outputs can be established using standard statistical methods.

Various methods can be utilized for quantifying the presence of the selected markers. For measuring the amount of a molecule that is present, a convenient method is to label the molecule with a detectable moiety, which may be fluorescent, luminescent, radioactive, or enzymatically active. Fluorescent and luminescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g., by expressing them as green fluorescent protein chimeras inside cells.

Output variables may be measured by immunoassay techniques such as, immunohistochemistry, radioimmunoassay (RIA) or enzyme linked immunosorbance assay (ELISA) and related non-enzymatic techniques. These techniques utilize specific antibodies as reporter molecules that are particularly useful due to their high degree of specificity for attaching to a single molecular target. Cell based ELISA or related non-enzymatic or fluorescence-based methods enable measurement of cell surface parameters. Readouts from such assays may be the mean fluorescence associated with individual fluorescent antibody-detected cell surface molecules or cytokines, or the average fluorescence intensity, the median fluorescence-intensity, the variance in fluorescence intensity, or some relationship among these.

The results of screening assays may be compared to results obtained from reference compounds, concentration curves, controls, etc. The comparison of results is accomplished by the use of suitable deduction protocols, AI systems, statistical comparisons, etc.

A database of reference output data can be compiled. These databases may include results from known agents or combinations of agents, as well as references from the analysis of cells treated under environmental conditions in which single or multiple environmental conditions or parameters are removed or specifically altered. A data matrix may be generated, where each point of the data matrix corresponds to a read-out from a output variable, where data for each output may come from replicate determinations, e.g., multiple individual cells of the same type.

The readout may be a mean, average, median or the variance or other statistically or mathematically derived value associated with the measurement. The output readout information may be further refined by direct comparison with the corresponding reference readout. The absolute values obtained for each output under identical conditions will display a variability that is inherent in live biological systems and also reflects individual cellular variability as well as the variability inherent between individuals.

Alternative in vivo uses for the device include implantation into a subject for experimental studies, to provide assistance for impaired functions, to augment natural functions, or to provide extra capabilities.

While the invention has been described in detail and with reference to specific examples thereof, it will be apparent to one skilled in the art that various changes and modifications can be made therein without departing from the spirit and scope thereof.

### REFERENCES

1. Abbot A. Biology's new dimension. Nature 2003; 424: 870-872.
2. Andersson H, van den Berg A. Microfluidic devices for cellomics: a review. Sensors and Actuators B 2003; 92: 315-325.
3. Yi C, Li C—W, Ji S, Yang M. Microfluidics technology for manipulation and analysis of biological cells. Analytica Chimica Acta 2006; 560:1-23.
4. Madou M. Fundamentals of Microfabrication. CRC Press: New York, 2002.
5. Tabeling P. Introduction to Microfluidics. Oxford University Press: New York, 2005.

What is claimed is:

1. A microfluidic system for monitoring or detecting a change in a parameter of an input substance, the microfluidic system comprising:
a microfluidic device, wherein the microfluidic device comprises (a) a cover platform having an inlet for delivery of an input substance and an outlet for removal of an output substance, (b) a substrate platform having (i) a tissue chamber in a shape of a depression in a substrate body of the substrate platform and (ii) a tissue analog having a vessel structure mimicking naturally occurring vessel network in a tissue analog three-dimensional construct comprising cells mixed with a tissue analog matrix, (c) a first microfluidic channel in fluid communication with the inlet for delivery of the input substance and the tissue chamber and (d) a second microfluidic channel in fluid communication with the outlet for removal of the output substance, provided that the substrate platform and the cover platform are superimposed to form a sealed assembly;
an input substance unit; and
optionally a pumping assembly and a detecting unit.

2. The microfluidic system of claim 1, wherein the substrate platform comprises the first microfluidic channel and the second microfluidic channel in fluid communication with the tissue chamber.

3. The microfluidic system of claim 1, wherein the input substance is filled at least partially the vessel network of the tissue analog.

4. The microfluidic system of claim 1, wherein the cover platform comprises the first microfluidic channel and the second microfluidic channel in fluid communication with the tissue chamber.

5. The microfluidic system of claim 1, wherein at least one of the cover platform or the substrate platform comprises a surface with an improved hydrophilicity.

6. The microfluidic system of claim 1, wherein at least one of the cover platform or the substrate platform are made of a polymer, glass, a ceramic, a metal, an alloy, or a combination thereof.

7. The microfluidic system of claim 1, wherein the cover platform is made of a plasma treated glass and the substrate platform is made of a plasma treated biologically-compatible polymer composed of a plurality of siloxane units.

8. The microfluidic system of claim 1, wherein the tissue analog matrix comprises hydrogel.

9. The microfluidic system of claim 1, wherein the tissue analog is at least one of heart, stomach, kidney, intestine, lung, liver, fat, bone, cartilage, skeletal muscle, smooth muscle, cardiac muscle, bone marrow, muscle, brain, and pancreas.

10. The microfluidic system of claim 1, comprising a plurality of tissue chambers and microfluidic channels.

11. A method for monitoring or detecting a change in a parameter of an input substance, the method comprising:
providing a microfluidic system of claim 1;
providing the input substance unit comprising the input substance;
directing the input substance into the microfluidic device, wherein the input substance flows through the inlet for delivery of the input substance and the first microfluidic channel into the vessel network in the tissue analog;
removing the output substance from the microfluidic device via the second microfluidic channel and the outlet for removal of the output substance; and
obtaining at least a portion of the input substance prior to entry into the vessel network and at least a portion of the output substance after exiting the vessel network and thereby monitoring or detecting a change in the parameter of the input substance.

12. The method of claim 11, wherein the input comprises a drug and optionally a pharmaceutically acceptable carrier.

13. The method of claim 12, wherein said monitoring or detecting the change in the parameter of the input substance comprises collecting the output comprising a metabolite having a detectable parameter; detecting the detectable parameter; and correlating the detectable parameter to at least the extent and rate of metabolism.

14. A method of making the microfluidic system of claim 1, the method comprising:
fabricating the cover platform comprising a cover body, an inlet port, an inlet opening, an outlet port, an outlet opening, and optionally microfluidic channels using microfabrication techniques;
fabricating the substrate platform comprising a substrate body, a tissue chamber, a first microfluidic channel and a second microfluidic channel wherein each microfluidic channel is in fluid communication with an input entry compartment and an output removal compartment, provided that each of the tissue chamber, the first microfluidic channel, the second microfluidic channel, the input entry compartment, and the output removal compartment represent indentations or depressions in the substrate body;
plasma treating the substrate platform and the cover platform;
making the tissue analog having the vessel structure mimicking naturally occurring vessel network in the tissue analog three-dimensional construct comprising cells mixed with the tissue analog matrix by using a bioprinting freeform fabrication process for a layer-by-layer deposition of the tissue analog matrix comprising cells;
forming the microfluidic device by superimposing the cover platform with the substrate platform such that the first microfluidic channel and the second microfluidic channel are in fluid communication with the tissue chamber, the an inlet port, the an outlet port, and the vessel structure; and
sealing the microfluidic device to provide the sealed assembly such that a flow of a substance can be conducted by engaging at least the inlet port, the first microfluidic channel, the second microfluidic channel, the vessel structure, and the outlet port and thereby making the microfluidic system.

15. The method of claim 14, wherein the tissue analog matrix comprises hydrogel.

16. The method of claim 14, wherein the cover platform comprises microfluidic channels etched in the cover body.

17. The method of claim 14, wherein at least one of the cover platform or the substrate platform are made of a polymer, glass, a ceramic, a metal, an alloy, or a combination thereof.

18. The method of claim 17, wherein the cover platform is made of a plasma treated glass and the substrate platform is made of plasma treated biologically-compatible polymer composed of a plurality of siloxane units.

19. The method of claim 14, wherein the tissue analog is at least one of heart, stomach, kidney, intestine, lung, liver, fat, bone, cartilage, skeletal muscle, smooth muscle, cardiac muscle, bone marrow, muscle, brain, and pancreas.

20. The method of claim 14, further comprising connecting a pumping assembly and a detecting unit to the microfluidic device.
